(19) United States
(12) Patent Application Publication
(10) Pub. No.: US 2012/0067344 A1
(43) Pub. Date: Mar. 22, 2012

(54) RESPIRATORY MASK, RESPIRATORY MASK ARRANGEMENT, RESPIRATION SYSTEM AND PACKAGING UNIT

(75) Inventor: Angelika Koschany, Munchen (DE)

(73) Assignee: MEDICAL INNOVATIONS GMBH, Puchheim (DE)

(21) Appl. No.: 13/232,356

(22) Filed: Sep. 14, 2011

(30) Foreign Application Priority Data
Sep. 14, 2010 (DE) 10 2010 040 771.2

(51) Int. Cl. A61M 16/06; A61M 16/00

(52) U.S. Cl. 128/201.22; 128/207.13; 128/206.25

(57) ABSTRACT

A respiratory mask for artificially ventilating a patient, having: a nose receiving chamber; first and second fastening tabs each being arranged on respective nose wing portions of the nose receiving chamber and a third fastening tab being arranged on a nose root portion of the nose receiving chamber. A respiratory mask arrangement can include such a respiratory mask; a hat arranged on the head of the patient; and fastening means which cooperate with the fastening tabs and with the hat in order to fix the respiratory mask to the head of the patient. A respiration system can include such a respiratory mask arrangement; and a respiration device for producing a continuous positive airway pressure. A packaging unit can include such a respiratory mask having a border with a peripheral groove; a sealing means which is arranged in the peripheral groove and a protective film for covering the sealing means.

The present invention hereby makes reference to the specification of German utility model DE 202 06 692 U1, the entirety of the disclosure of which is incorporated herein by reference.

The present invention relates to a respiratory mask for artificially ventilating a patient, to a respiratory mask arrangement, to a respiration system and to a packaging unit. The respiratory mask is suitable in particular for premature babies and infants.

Respiratory masks are used inter alia for artificial ventilation in intensive care units. Such respiratory masks are produced in different sizes and shapes for typical nose shapes and sizes, so that a form-fitting respiratory mask is essentially available for every nose shape. The respiratory mask has a connection region for connection to a respiratory pump. The respiratory mask further has two fastening tabs, each of which is arranged on a side of the respiratory mask which is correspondingly associated with a wing of the patient's nose. The fastening tabs are fixed to the patient's head by a fastening means, such as a hook and loop strip.

However, the respiratory mask can easily be lifted away from the patient's face at the root of the patient's nose, for example when the patient moves. Respiratory air can hereby flow out of the respiratory mask. The functionality of the respiratory mask is not reliably ensured as a result. In particular, when the patient moves, a tensile load can be applied to connecting hoses between the respiratory mask and the respiratory pump, as a result of which the respiratory mask is at least partially lifted away from the patient's face. This is understandably to be avoided.

Starting therefrom, it is an object of the present invention to provide an improved respiratory mask which eliminates the above-mentioned drawbacks.

This object is achieved by a respiratory mask having the features of claim 1 and/or by a respiratory mask arrangement having the features of claim 6 and/or by a respiration system having the features of claim 9 and/or by a packaging unit having the features of claim 12.

Accordingly, there is provided a respiratory mask for artificially ventilating a patient, having: a nose receiving chamber; and at least three fastening tabs for fastening the respiratory mask to the patient, first and second fastening tabs each being arranged on respective nose wing portions of the nose receiving chamber and a third fastening tab being arranged on a nose root portion of the nose receiving chamber.

There is further provided a respiratory mask arrangement, having: such a respiratory mask; a hat arranged on the patient's head; and fastening means which cooperate with the fastening tabs and with the hat in order to fix the respiratory mask to the patient's head.

There is still further provided a respiration system, having: such a respiratory mask arrangement; and a respiration device for producing a continuous positive airway pressure.

There is still further provided a packaging unit, having: such a respiratory mask, the respiratory mask having a border with a peripheral groove; a sealing means, which is arranged in the peripheral groove at least in portions for sealing the respiratory mask against the patient's face; and a protective film for covering the sealing means until the respiratory mask is used.

The fundamental idea of the present invention is to provide a third fastening tab on the nose root portion of the nose receiving chamber, as a result of which, as compared with described solutions, it is always ensured that the respiratory mask is securely seated on the patient's face. The reliability and the wearing comfort of the respiratory mask are thereby increased significantly.

Advantageous embodiments and further developments are indicated in the dependent claims and the embodiments.

According to a preferred embodiment of the respiratory mask, a first fastening tab and a second fastening tab are arranged on a common first fastening axis, and a third fastening tab is arranged on a second fastening axis which is formed perpendicularly to the first fastening axis. By means of the perpendicular arrangement of the fastening axes relative to one another, it is ensured that the respiratory mask is securely seated on the patient's face, because the respiratory mask is fixed at three points which are as far apart as possible.

According to a further preferred embodiment of the respiratory mask, it has a peripheral border for resting on the patient's face. The contact area of the respiratory mask on the patient's face is hereby enlarged, as a result of which the wearing comfort for the patient is again increased and the seal between the respiratory mask and the patient's face is improved.

According to a further preferred development of the respiratory mask, the border has a peripheral groove for receiving a sealing means, in particular a silicone gel, as a result of which the seal between the respiratory mask and the patient's face is further improved. The reliability of the respiratory mask is thereby increased.

According to a further preferred embodiment of the respiratory mask, the fastening tabs are arranged on the border. As a result, the respiratory mask is particularly simple to produce because, for example, a corresponding moulding tool has a less complex structure, as a result of which the costs of producing the respiratory mask are reduced.

According to a preferred embodiment of the respiratory mask arrangement, the fastening means are in the form of hook and loop strips. As a result, the respiratory mask can be fixed to the patient in a particularly quick, variable and convenient manner, which simplifies handling of the respiratory mask.

According to a further preferred embodiment of the respiratory mask arrangement, a respective fastening means cooperates with each ear portion of the hat and a fastening means cooperates with a forehead portion of the hat. The respiratory mask is hereby fixed both in the direction of the patient's ears and in the direction of the patient's forehead, as a result of which the respiratory mask is prevented from slipping.

According to a preferred embodiment of the respiration system, the hat has a fastening device on a forehead portion for fastening the respiration device to the hat. The respiration device is hereby secured to the hat, as a result of which the respiratory mask is reliably prevented from slipping due to a movement of the respiration device, for example when the patient moves.

According to a further preferred embodiment of the respiration system, the fastening device is in the form of a hook and loop device, as a result of which the respiration device can be fixed to the hat particularly quickly and conveniently. Handling of the respiration system is thereby simplified and accelerated.

According to a preferred embodiment of the packaging unit, the sealing means is in the form of silicone gel. Excellent tolerance of the patient towards the sealing means is thereby achieved, as a result of which the comfort of the patient is increased.

The present invention is explained hereinbelow with reference to preferred embodiments and the accompanying drawings, in which:

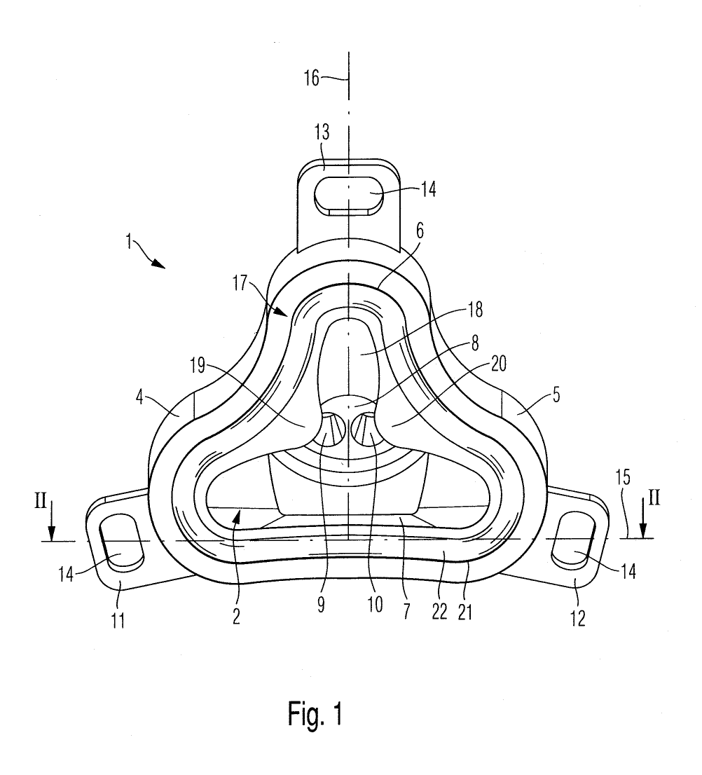

FIG. 1 is a rear view of a preferred embodiment of a respiratory mask;

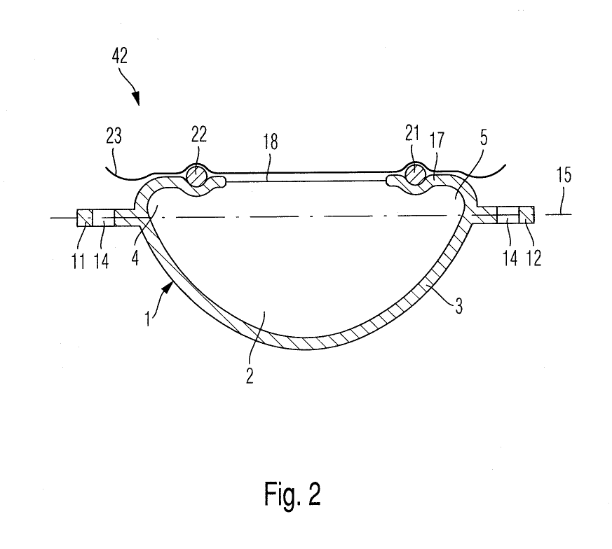

FIG. 2 is a sectional view of the respiratory mask taken along line II-II in FIG. 1;

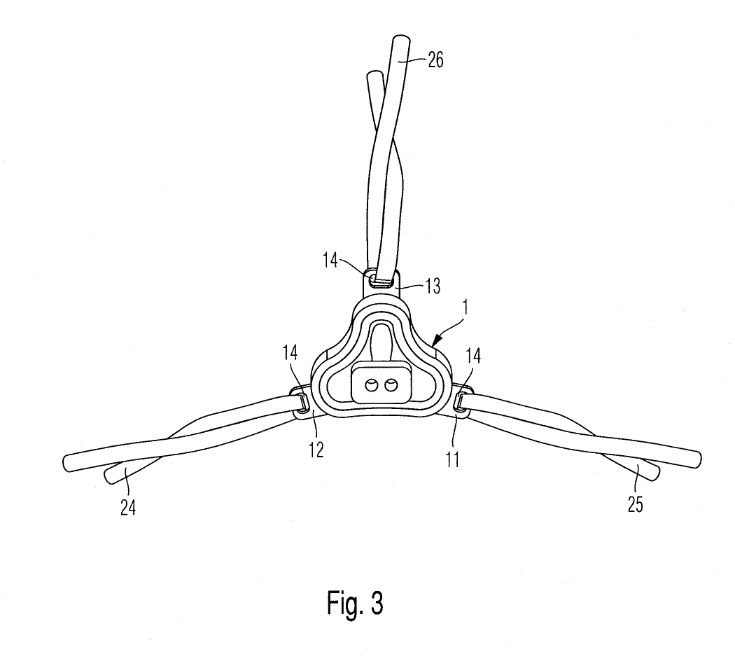

FIG. 3 is a front view of the respiratory mask according to FIG. 1 with fastening means;

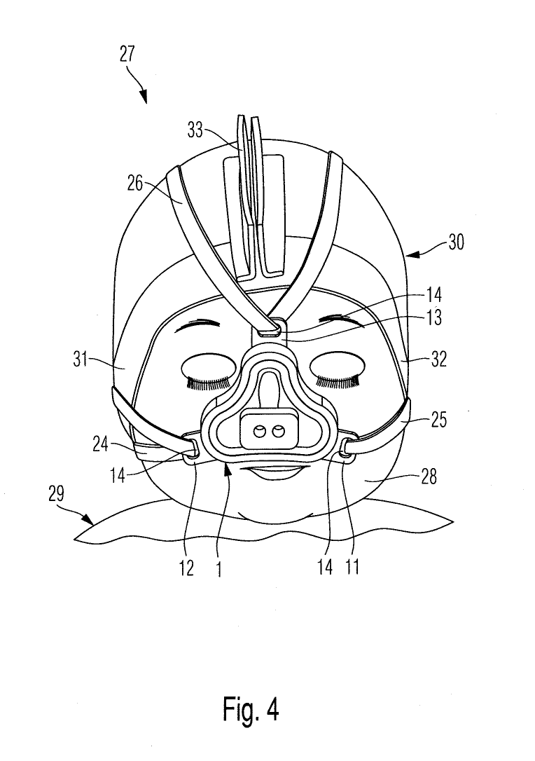

FIG. 4 is a front view of a preferred embodiment of a respiratory mask arrangement;

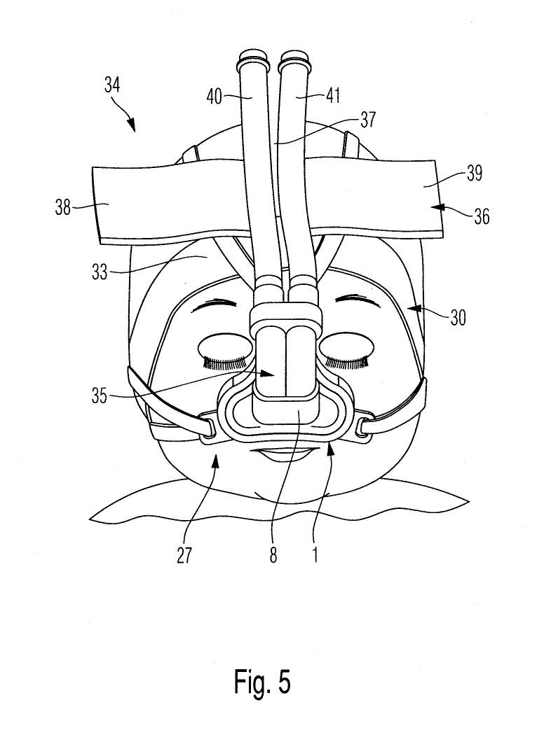

FIG. 5 is a front view of a preferred embodiment of a respiration system; and

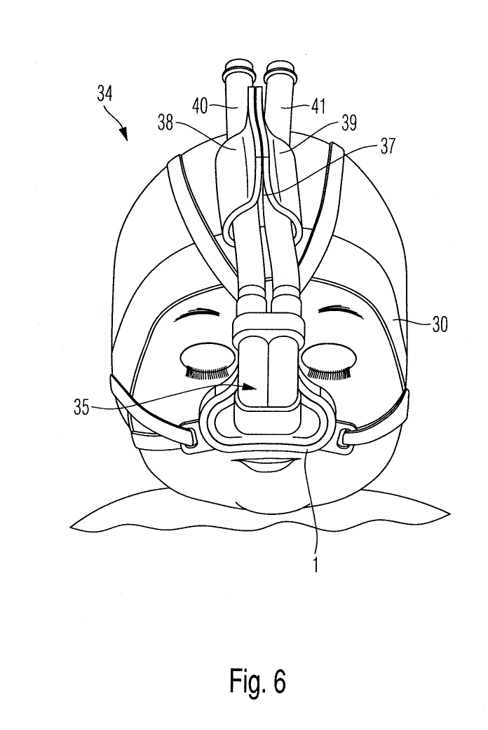

FIG. 6 is a further front view of the respiration system according to FIG. 5.

In the figures, like reference numerals denote like or functionally equivalent elements.

FIGS. 1 and 2, to which reference is made simultaneously hereinbelow, show a rear view and a sectional view of a preferred embodiment of a respiratory mask 1 for artificially ventilating a patient, in particular a baby or infant.

The respiratory mask 1 preferably has a nose receiving chamber 2 for at least partially receiving a patient's nose. In particular, the nose receiving chamber 2 is matched to a geometry of the patient's nose. A geometrical shape and a volume of the nose receiving chamber 2 is preferably such that, when the respiratory mask 1 is placed on the patient's face, the nose receiving chamber 2 is filled preferably completely or almost completely by the nose, in particular the respiratory mask 1 encloses the nose in a form-fitting manner at least in portions. A wall 3 of the nose receiving chamber 2 is preferably in such a form that it does not press on the nose. The respiratory mask 1 is, for example, provided in different shapes and sizes. The wall 3 of the nose receiving chamber 2 is preferably formed from a particularly soft and flexible material so that it adapts to the patient's nose. Particularly suitable materials for the wall 3 are soft and resilient silicone and/or polyurethane plastics. The respiratory mask 1 is preferably in the form of a disposable article.

The nose receiving chamber 2 preferably has two nose wing portions 4, 5, each of which is associated with a respective nose wing of the patient's nose, and a nose root portion 6, which is associated with a nose root of the patient's nose. The nose receiving chamber 2 of the respiratory mask 2 further has a nose tip portion 7, which is associated with a nose tip of the patient's nose.

In the nose tip portion 7 of the nose receiving chamber 2 there is preferably provided a connecting piece 8 for connecting a respiration device to the respiratory mask 1. The respiration device produces a continuous positive airway pressure, that is to say the patient breathes against an overpressure. Such a respiration device is described, for example, in the specification of German utility model DE 202 06 692 U1. The connecting piece 8 preferably has approximately the form of a cuboid which has, for example, two openings 9, 10 arranged side by side. The connecting piece 8 is preferably formed in one piece with the wall 3 of the nose receiving chamber 2. The openings 9, 10 supply fresh air to the patient and carry used respiratory air away from the patient. For example, the opening 9 is in the form of the gas inlet 9 and the opening 10 is in the form of the gas outlet 10 of the respiratory mask 1. By means of the respiration device, gas flows continuously through the nose receiving chamber 2 and carbon dioxide contained in the air exhaled by the patient is washed out.

The respiratory mask 1 further has fastening tabs 11, 12, 13. The number of fastening tabs is arbitrary, but the respiratory mask 1 preferably has at least three fastening tabs 11, 12, 13. Each fastening tab 11, 12, 13 has an opening 14 for the passage of a fastening means. The opening 14 is, for example, in the form of an ellipse, a circle, a rounded rectangle or the like. The fastening tabs 11, 12, 13 are preferably formed in one piece with the wall 3 of the nose receiving chamber 2. For example, the fastening tabs 11, 12, 13 are set back slightly relative to a contact surface 17 of the respiratory mask 1, as a result of which improved contact pressure of the respiratory mask 1 on the patient's face is obtained. Preferably, first and second fastening tabs 11, 12 are associated with respective nose wing portions 4, 5 of the nose receiving chamber 2 and, in particular, are arranged thereon. For example, the fastening tabs 11, 12 are arranged on an outer face of the wall 3 on the corresponding nose wing portion 4, 5. For example, the first fastening tab 11 is associated with a first nose wing portion 4 and the second fastening tab 12 is associated with a second nose wing portion 5. The first fastening tab 11 and the second fastening tab 12, in particular the respective openings 14, are arranged, for example, on a common first fastening axis 15 of the respiratory mask 1. A third fastening tab 13 is preferably associated with the nose root portion 6 of the nose receiving chamber 2 and in particular is arranged thereon. For example, the fastening tab 13 is arranged on the outer face of the wall 3 on the nose root portion 6. The third fastening tab 13, or the opening 14 in the third fastening tab 13, is preferably arranged on a second fastening axis 16 formed perpendicularly to the first fastening axis 15. The fastening tabs 11, 12, 13, or the respective openings 14 in the fastening tabs 11, 12, 13, thus preferably form substantially the corners of an isosceles or equilateral triangle. The fastening tabs 11, 12, 13 extend away from the nose receiving chamber 2 into the area surrounding the respiratory mask 1.

The contact surface 17 of the respiratory mask 1 is preferably in the form of a peripheral border 17 which at least partially seals the nose receiving chamber 2 to the patient's face. By means of the peripheral border 17, the respiratory mask 1 rests flat against the patient's face. The peripheral border 17 preferably seals the nose receiving chamber 2 to the patient's face apart from an approximately T-shaped opening 18. The border 17 has, for example, sealing tabs 19, 20 which are flexible and rest on the nose wings of the patient's nose. The border 17 is preferably formed in one piece with the wall 3 of the nose receiving chamber 2. The border 17 allows the respiratory mask 1 to be sealed against the patient's face and increases the wearing comfort for the patient owing to the enlarged contact area. The fastening tabs 11, 12, 13 can be provided on the border 17.

The respiratory mask 1 further has a peripheral groove 21 provided in the peripheral border 17. The groove 21 is provided in the border 17 in the form of, for example, a semicircular depression or indentation. The groove 17 preferably serves to receive a sealing means 22, in particular a silicone gel 22, at least in portions. The sealing means 22 seals the respiratory mask 1 against the patient's face. In order to protect the sealing means 22 before the respiratory mask 1 is used, it is covered, for example, with a removable protective film 23. The protective film 23 is in the form of polyethylene film, for example. Preferably, the protective film 23 also closes the nose receiving chamber 2 completely before the respiratory mask 1 is used. As a result, the nose receiving chamber 2 is protected from dirt before the respiratory mask 1 is used. The respiratory mask 1 with the sealing means 22 and the protective film 23 forms, for example, a packaging unit 42 or packaging arrangement 42, which is obtainable as auxiliary equipment for a respiration device.

FIG. 3 shows a front view of the respiratory mask 1 according to FIGS. 1 and 2. Fastening means 24, 25, 26 for fastening the respiratory mask 1 to the patient are passed through the respective openings 14 in the fastening tabs 11, 12, 13. The fastening means 24, 25, 26 are preferably in the form of hook and loop strips 24, 25, 26. Alternatively, the fastening means 24, 25, 26 can also have snap fasteners or any other fastenings which can be released again. The hook and loop strips 24, 25, 26 are preferably in such a form that each hook and loop strip 24, 25, 25 has at least one fleece portion or loop portion and at least one hook portion for engaging in a corresponding fleece portion or loop section. The corresponding fleece portion or loop portion can be provided on the same hook and loop strip 24, 25, 25 or on a different component, for example an article of clothing. The hook and loop strips 24, 25, 26 can be provided with hook portions and/or fleece portions or loop portions on one side or on both sides.

FIG. 4 shows a respiratory mask arrangement 27 having a respiratory mask 1 according to FIGS. 1 to 3 and a hat 30 arranged on the head 28 of the patient 29. The hat 30 can be in the form of a disposable article or a re-usable, in particular sterilisable, article. Preferably, the hat 30 is provided in different sizes and shapes in order to achieve an optimal fit on the head 28 of the patient 29.

For fixing the respiratory mask 1 to the head 28 of the patient 29, the fastening means 24, 25, 26 cooperate both with the fastening tabs 11, 12, 13 of the respiratory mask 1 and with the hat 30. The fastening means 24, 25, 26 are passed through the respective openings 14 in the fastening tabs 11, 12, 13. The fastening means 24, 25, 26 are preferably in functional engagement with the fabric of the hat 30. For example, portions of the hat 30 or the whole of the hat 30 is/are formed of a material which, for example, can be brought into functional engagement with hook portions of the fastening means 24, 25, 26. For example, the hat 30 is formed of a fleece material or loop material which is complementary to the hook portions of the fastening means 24, 25, 26. The respiratory mask 1 is thereby securely fixed to the head 28 of the patient 29. Preferably, a fastening means 24, 25 cooperates with a respective ear portion 31, 32 and with a respective fastening tab 11, 12. The fastening means 26 preferably cooperates with a forehead portion 33 and with the fastening tab 13 associated with the nose root portion 6 of the respiratory mask 1. It is hereby ensured that the respiratory mask 1 is also securely seated on the nose root of the nose of the patient 29, as a result of which the reliability of the respiratory mask is increased.

FIG. 5 shows a respiration system 34 having a respiratory mask arrangement 27 according to FIG. 4. The respiration system 34 further has a respiration device 35 for producing a continuous positive airway pressure. The respiration device 35 is connected to a respiratory pump (not shown), for example via connecting hoses. The respiration device 35 cooperates with the connecting piece 8 of the respiratory mask 1. Such a respiration device 35 is described, for example, in the specification of German utility model DE 202 06 695 U1.

The hat 30 preferably has a fastening device 36 arranged on its forehead portion 33 for fastening the respiration device 35 to the hat 30. The fastening device 36 is preferably in the form of a hook and loop device 36. Alternatively, the fastening device 36 can also have a snap fastening, string fastening, hook fastening or any other fastening which can be released again. The hook and loop device 35 has, for example, a central double-sided fleece or loop strip 37 and two hook strips 38, 39 arranged on either side of the fleece strip 37. The fastening device 36 is, for example, sewn or adhesively bonded to the forehead portion 33 of the hat 30. The fastening device 36 can also be in such a form that it is detachable from the hat 30.

FIG. 5 shows the fastening device 36 in an open state. In order to fasten the respiration device 35 to the hat 30, the central fleece strip 37 of the fastening device 36, for example, is passed between two connecting hoses 40, 41 of the respiration device 35 and the respective hook strips 38, 39 are passed around the connecting hoses 40, 41 and placed in cooperation with the fleece strip 37. The respiration device 35 is thereby securely fixed to the head 28 of the patient 29, as shown in FIG. 6.

### LIST OF REFERENCE NUMERALS

1 Respiratory mask
2 Nose receiving chamber
3 Wall
4 Nose wing section
5 Nose wing section
6 Nose root section
7 Nose tip section
8 Connecting piece
9 Opening
10 Opening
11 Fastening tab
12 Fastening tab
13 Fastening tab
14 Opening
15 Fastening axis
16 Fastening axis
17 Contact surface
18 Opening
19 Sealing tab
20 Sealing tab
21 Groove
22 Sealing means
23 Protective film
24 Fastening means
25 Fastening means
26 Fastening means
27 Respiratory mask arrangement
28 Head
29 Patient
30 Hat
31 Ear section
32 Ear section
33 Forehead section
34 Respiration system
35 Respiration device
36 Fastening device
37 Fleece strip
38 Hook strip
39 Hook strip
40 Connecting hose
41 Connecting hose
42 Packaging unit

1. Respiratory mask for artificially ventilating a patient, having:
a nose receiving chamber; and at least three fastening tabs for fastening the respiratory mask to the patient, first and second fastening tabs each being arranged on respective nose wing portions of the nose receiving chamber and a third fastening tab being arranged on a nose root portion of the nose receiving chamber.

2. Respiratory mask according to claim 1, wherein the first fastening tab and the second fastening tab are arranged on a common first fastening axis, and in that the third fastening tab is arranged on a second fastening axis formed perpendicularly to the first fastening axis.

3. Respiratory mask according to claim 1, wherein the respiratory mask has a peripheral border for resting on the face of the patient.

4. Respiratory mask according to claim 3, wherein the border has a peripheral groove for receiving a sealing means, in particular a silicone gel.

5. Respiratory mask according to claim 3, wherein the fastening tabs are arranged on the border.

6. Respiratory mask arrangement, having:
a respiratory mask according to any one of the preceding claims;
a hat arranged on the head of the patient; and fastening means which cooperate with the fastening tabs and with the hat in order to fix the respiratory mask to the head of the patient.

7. Respiratory mask arrangement according to claim 6, wherein the fastening means are in the form of hook and loop strips.

8. Respiratory mask arrangement according to claim 6, wherein respective fastening means each cooperate with a respective ear portion of the hat, and a fastening means cooperates with a forehead portion of the hat.

9. Respiration system, comprising:
the respiratory mask arrangement according to claim 6; and
further comprising a respiration device for producing a continuous positive airway pressure.

10. Respiration system according to claim 9, wherein the hat has on a forehead portion a fastening device for fastening the respiration device to the hat.

11. Respiration system according to claim 10, wherein the fastening device is in the form of a hook and loop device.

12. Packaging unit, comprising:
the respiratory mask according to claim 1, the respiratory mask having a border with a peripheral groove;
a sealing means which is arranged in the peripheral groove at least in portions in order to seal the respiratory mask against the face of the patient; and a protective film for covering the sealing means until the respiratory mask is used.

13. Packaging unit according to claim 12, wherein the sealing means is in the form of silicone gel.

14. Respiratory mask according to claim 2, wherein the respiratory mask has a peripheral border for resting on the face of the patient.

15. Respiratory mask according to claim 4, wherein the fastening tabs are arranged on the border.

16. Respiratory mask arrangement according to claim 7, wherein respective fastening means each cooperate with a respective ear portion of the hat, and a fastening means cooperates with a forehead portion of the hat.

17. Respiration system, comprising:
the respiratory mask arrangement according to claim 7; and further comprising a respiration device for producing a continuous positive airway pressure.

18. Respiration system, comprising:
the respiratory mask arrangement according to claim 8; and further comprising a respiration device for producing a continuous positive airway pressure.

19. Packaging unit, comprising:
the respiratory mask according to claim 2, the respiratory mask having a border with a peripheral groove;
a sealing means which is arranged in the peripheral groove at least in portions in order to seal the respiratory mask against the face of the patient; and a protective film for covering the sealing means until the respiratory mask is used.

20. Packaging unit, comprising:
the respiratory mask according to claim 3, the respiratory mask having a border with a peripheral groove;
a sealing means which is arranged in the peripheral groove at least in portions in order to seal the respiratory mask against the face of the patient; and a protective film for covering the sealing means until the respiratory mask is used.

21. Packaging unit, comprising:
the respiratory mask according to claim 4, the respiratory mask having a border with a peripheral groove;
a sealing means which is arranged in the peripheral groove at least in portions in order to seal the respiratory mask against the face of the patient; and a protective film for covering the sealing means until the respiratory mask is used.

22. Packaging unit, comprising:
the respiratory mask according to claim 5, the respiratory mask having a border with a peripheral groove;
a sealing means which is arranged in the peripheral groove at least in portions in order to seal the respiratory mask against the face of the patient; and a protective film for covering the sealing means until the respiratory mask is used.

23. Respiratory mask according to claim 2, wherein the respiratory mask has a peripheral border for resting on the face of the patient.
